(19) United States
(12) Patent Application Publication
(10) Pub. No.: US 2015/0352344 A1
(43) Pub. Date: Dec. 10, 2015

(54) Back lotioner-oil

(71) Applicant: Tonya Vernita Cotton, Decatur, GA (US)

(72) Inventor: Tonya Vernita Cotton, Decatur, GA (US)

(21) Appl. No.: 11/483,801

(22) Filed: Aug. 14, 2006

(51) Int. Cl. A61M 35/00; A45D 40/26; A45D 34/04

(52) U.S. Cl. CPC A61M 35/003; A45D 34/04; A45D 40/26

(57) ABSTRACT

The back lotioner is designed to administer lotions, oils, sun screen and any other lubricant to your back. There is always a moment in time where you do not have enough arms to reach your back, and that is what is my back lotioner is for. Even when you have medication to administer to your back, for those that may have eczema or psoriasis the back lotioner will do it. The back lotioner will help keep dry patches from formulating on your back and eliminating those awkward moments where you do not want to ask anyone to lotion your back.

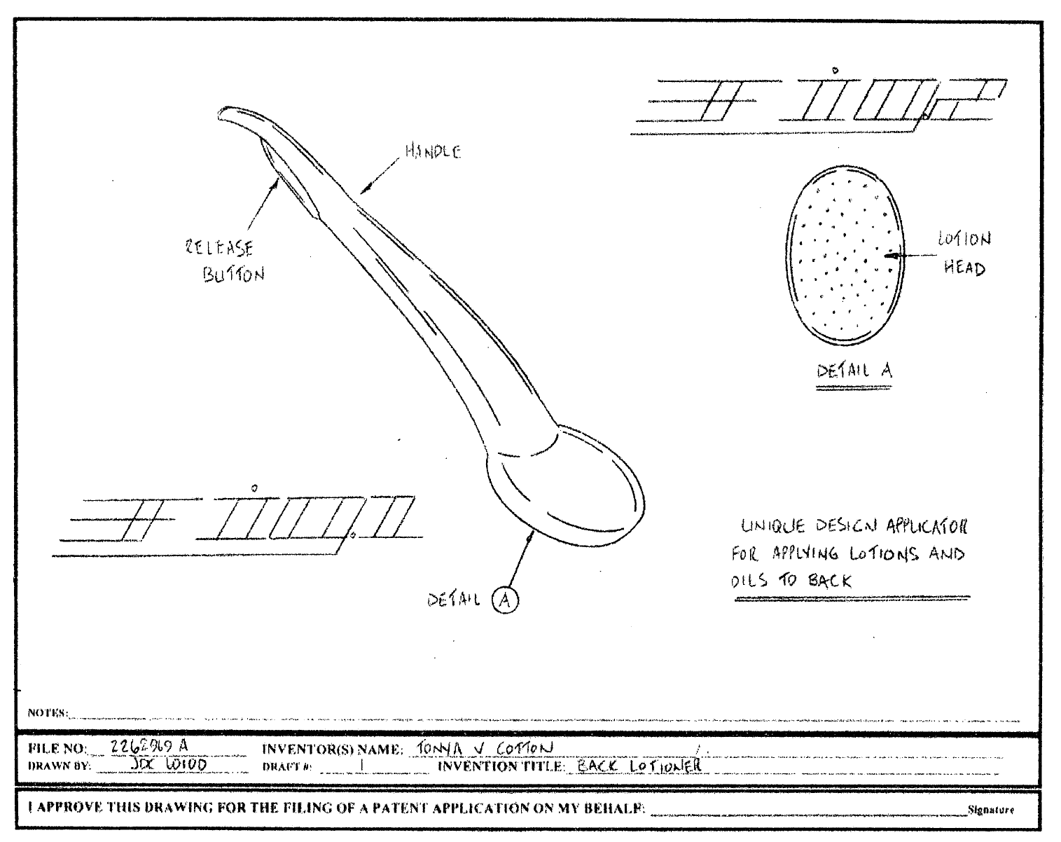

### INVENTION DESCRIPTION AND FUNCTIONAL OVERVIEW

To facilitate one's comprehension and understanding of a new product, that idea must be described not only as it exists as an answer to a subsisting, continuing problem, but also as it functions physically. Based upon the Inventor's description and our outside research, we will describe the basic overall features, shape, design, and materials used to comprise BACK LOTIONER.

In the spirit of progress, PTI is introducing a concept known as BACK LOTIONER, which would be capable of applying lotions to every inch of a person's skin. This product will eliminate the unnatural positions people currently put their selves in when attempting to apply skin moisturizers, sunscreens, or sun tanning oils to the hard to reach areas of their body.

BACK LOTIONER would be produced from a durable plastic material. ABS (Acrylonitrile-Butadiene-Styrene) is a type of molded plastic that might be considered the plastic of choice for BACK LOTIONER. Its high rigidity and impact strength, excellent abrasion and moisture resistance, and its resistance to most household chemicals characterize ABS. ABS is easily thermoformed and lends itself remarkably well to mold detail.

The plastic material would be molded into a long, slightly curved, hollow handle. The interior portion of the handle would be designed to hold a measured amount of lotion or oil. The base of the handle would be slightly narrower than the handle's body and equipped with a small button. The button would be connected to an interior pump system that would force the oil or lotion from the handle and out onto the product's applicator.

BACK LOTIONER'S applicator would be oval shaped and covered with numerous, small circular shaped openings. The openings would serve as a passage through which the lotion would be dispensed. The applicator would be covered with a pad, also equipped with the same pattern of openings, to soften the contact between the applicator head and the user's skin.

BACK LOTIONER would be approximately two and one-half feet in length and three inches wide at its widest point.

BACK LOTIONER would be extremely easy to use. The user would simply press the button to force a measured amount of lotion or oil onto the applicator head. The individual would then grip the handle and move the applicator over the desired area of skin to evenly and thoroughly apply the desired product.

### PROPOSED MODIFICATIONS

In order to fully appreciate the benefits and need of an innovation, one should consider any possible modifications that would arise from the use of the idea.

PTI, therefore, shall suggest other applications of BACK LOTIONER that would merit future consideration and study. The scope of this review may include the use of materials other than those specified by the Inventor. In addition, this review may support the use of additional materials, color schemes, diversified applications, broader consumer outlets, or other possible shape and design alterations that may enhance the utility and salability of the Invention. All in all, we shall attempt to identify any variations that would further define the scope of the idea.

One must remember, however, that these modification suggestions are limited within the latitude of the Invention. Some Inventions lend themselves easily to future applications, i.e. different shapes, materials, color schemes, etc. Other Inventions, however, are geared to a specific group of consumers, to one particular design, or to a strict manufacturing process. As a result, any suggested modifications to the Invention may be limited within the Invention's scope.

The following modifications are suggested to augment the usefulness and popularity of BACK LOTIONER. It should be noted that neither these nor any other suggested modifications or variations of your Invention are included in your Preliminary Patent Art.
Modify BACK LOTIONER to be produced with a telescopic handle in order to allow the consumer to adjust the length of the handle to meet his or her personal needs.
Modify BACK LOTIONER to be produced in a variety of colors to make product more aesthetically appealing.
Market BACK LOTIONER with a tote bag to help keep the applicator clean between uses.
Modify BACK LOTIONER to be produced with disposable applicator pads as well as reusable pads.

In addition, detailed diagrams and technical specifications remain to be created. Nonetheless, there is a clear idea of the Inventor's intent and goals in submitting this design.

### PRODUCTION CONSIDERATIONS

We now turn to an examination of BACK LOTIONER through a study of production considerations. We also shall briefly discuss the need of engineering guidance, materials, styles, sizes, etc.

Companies interested in a new product consider many factors that influence the production of a product. Technology, materials, labor, overhead, and financial commitment are some of the key factors involved in this decision-making process.

Our review of BACK LOTIONER indicates that it can be manufactured using standard production of similar products. Materials, processes, and even construction techniques would be similar, with adaptations made for the specific design of BACK LOTIONER. Variations on the basic model would vary in price depending upon the cost of their specific components. The materials submitted by TONYA V. COTTON for BACK LOTIONER appear to meet the standards set by the U.S. Government, i.e., OSHA, EPA, and UL.

In today's environment, the materials that are used in factories are of considerable importance. With the public awareness of clean air, clean water, and pollution, the laws governing these areas are strictly enforced. Companies, especially in the health and beauty industry, are very cautious when it comes to using materials not environmentally approved. The materials recommended by the Inventor for BACK LOTIONER do not appear to be problematic.

The components of BACK LOTIONER utilize standard production technology. No new research or development in this area is anticipated, nor is major retooling or materials expense expected. In effect, this provides the company with lower start-up costs, which is a major consideration for product development.

The basic concept behind BACK LOTIONER appears to be sound, and the product idea is workable. The basic materials are readily available, and the concept would appeal to a vast variety of people. The design features in BACK LOTIONER seem well within the limits of present-day technology.

Given the previous description of how the Invention would function and taking into consideration the questions facing further development of BACK LOTIONER, it is our tentative judgment that the idea is based on sound principles and that a production version of this item could be developed to perform generally as the Inventor states.

Potential problems that might arise during the development of BACK LOTIONER should be amenable to resolution through normal product testing and refinement processes, after which we would anticipate that the product could be produced routinely. However, we also recommend that an interested company should be allowed to do their own form of testing and marketing and to provide modification suggestions.

Overall, since this product appears to be well thought out and potentially workable, there should be little added research or development required for the production phase of BACK LOTIONER.

The design is innovative and yet relatively simple. Existing production processes and techniques will suffice without resorting to new technology. Based upon our review of the material submitted by TONYA V. COTTON, it is our tentative conclusion that this product can be manufactured from conventional and commercially available materials with existing production technology.

### HISTORICAL DEVELOPMENT AND ADVANTAGES

To assess any new product's future acceptance and popularity, one should research the background and the need of such an innovation. This section addresses the background of the idea's conception and the problem(s) it is designed to ease or eliminate.

Millions of people across the world experience a common problem when attempting to apply lotions to their backs. Their arm and shoulder muscles limit the area of their backs that they can make contact with, which is only the top and base. The inflexibility of these muscles prevents people from being able to apply these products to the middle of their backs. Despite the amount of twisting and bending they do, they are generally unsuccessful and this area is neglected.

The problem of having to make contact with a person's back is intensified when an individual suffers with some form of physical disability, including arthritis, bursitis, weak muscles or other disabilities that decrease a person's range of motion. These individuals often have to rely on assistance with this seemingly simple task. Asking for assistance is not only humiliating, but also helps to further decrease the amount of self-esteem they have left.

The middle of a person's back is often not the only difficult area for some individuals to reach. Individuals who are obese, pregnant, or who have limited physical abilities may find making contact with their legs and feet equally as difficult as making contact with the middle of their backs.

BACK LOTIONER is designed by TONYA V. COTTON to address this problem. This product will enable individuals to apply lotion to every inch of their skin because of the simplicity of the process. BACK LOTIONER will reduce the time the process of applying lotion generally consumes, thus encouraging individuals to provide their skin with the moisturizer it needs to be healthy. It will also eliminate the mess involved with using one's hands to apply the lotion. BACK LOTIONER may also be used to apply sun tanning oils and baby oil. BACK LOTIONER would be simple to use and inexpensive to produce.

BACK LOTIONER could be marketed to any average household.

The purchase of such an item as BACK LOTIONER is dependent upon the level of discretionary purchasing power, which is the portion of a consumer's income remaining after necessities such as housing, transportation, and food have been accounted for. About 60% of after-tax income is spent on these fixed expenses. The remaining 40% is spent on variables. According to the U.S. Census Bureau's March 2000 Current Population Survey (most recent Government statistics), disposable income totals nearly $7,393.2 billion, which is about 19% of the total after-tax income received by the average household. This is an increase of 17% over 1996.

An overview of the economy of the U.S. in general and an analysis of the manufacturing industries associated with BACK LOTIONER in particular are appropriate in reviewing this product. According to the Journal of Business and Industrial Marketing, “Industrial firms must continue to create innovative products despite high new product introduction risk; they may otherwise face loss of market shares.” A survey of 900 companies found they would likely derive 33% of their profits over several years from new products.

The U.S. economy is unpredictable. There are varying times of growth and times of stagnation. Important consequences of this volatility include an increase/decrease in capital spending, resulting in greater/lower manufacturing capabilities and higher/lower consumer disposable income, which gives consumers the ability to spend more money or not on products they want, rather than just on products they need. These factors help to create the environment into which many new product ideas are effectively introduced.

1. Applicator with long handle and bottom oval shaped and covered with numerous, small circular shaped openings.
For Lotion and Oil, to be dispensed from.
The Applicator user would simply press the release button to force measured amount of Lotion or Oil into the applicator
head. The individual would then grip the handle and move applicator over the skin evenly and thoroughly apply the desired
product. As shown and described.
